(19) United States
(12) Patent Application Publication
(10) Pub. No.: US 2022/0157145 A1
(43) Pub. Date: May 19, 2022

(54) Daily Activity Sensor and System

(71) Applicant: VitalTech Properties, LLC, Plano, TX (US)

(72) Inventors: Anjan Panneer Selvam, Plano, TX (US); Peter Ianace, Frisco, TX (US)

(73) Assignee: VitalTech Properties, LLC, Plano, TX (US)

(21) Appl. No.: 17/440,926

(22) PCT Filed: Mar. 22, 2020
(86) PCT No.: PCT/US20/24102

§ 371 Date: Sep. 20, 2021

Related U.S. Application Data
(60) Provisional application No. 62/890,847, filed on Aug. 23, 2019.
(60) Provisional application No. 62/851,513, filed on May 22, 2019.
(60) Provisional application No. 62/822,274, filed on Mar. 22, 2019.
(60) Provisional application No. 62/822,381, filed on Mar. 22, 2019.
(60) Provisional application No. 62/822,434, filed on Mar. 22, 2019.

(51) Int. Cl. G08B 21/04; H04W 4/38; A61B 5/00; A61B 5/11

(52) U.S. Cl. CPC G08B 21/0423; H04W 4/38; G08B 21/043; A61B 5/746; A61B 5/6801; H04W 84/18; A61B 5/1117; A61B 5/1118; A61B 5/1113; A61B 5/0002; A61B 5/6887

(57) ABSTRACT

A system and method for monitoring activities of a user. Sensor measurements are performed utilizing a sensor array of sensor tags. User activities are detected utilizing the sensor array of the sensor tags. The user activities are communicated to a hub associated with the sensor array of sensor tags.

### PRIORITY

This application claims priority to U.S. Provisional Patent Application Nos. 62/822,274, 62/822,381, and 62/822,434 filed Mar. 22, 2019, U.S. Provisional Application No. 62/851,513 filed May 22, 2019, and U.S. Provisional Application No. 62/890,847 filed on Aug. 23, 2019, hereby incorporated by reference in their entirety.

### BACKGROUND

### I. Field of the Disclosure

The illustrative embodiments relate to sensors for determining user information. More specifically, but not exclusively, the illustrative embodiments relate to a system, method, sensor network, and sensors for monitoring and tracking activities of a user.

### II. Description of the Art

Many individuals in our country are entering the latter stages of their life. As a result, they are experiencing more health issues than they have experienced in the past. It is important for us as a society, including family, friends, and medical professionals to help ensure their well-being. It is particularly important to be watchful for negative events, such as a fall, stroke, heart attack, significant illness, deteriorating condition, or other negative health event. Without someone being physically present at all times, it may be difficult to know of or detect these negative events to best provide assistance, support, and medical help.

### BRIEF DESCRIPTION OF THE DRAWINGS

Illustrated embodiments are described in detail below with reference to the attached drawing figures, which are incorporated by reference herein, and where:

FIG. 1 is a pictorial representation of a sensor environment in accordance with an illustrative embodiment;

FIG. 2 is a block diagram of a sensor in accordance with an illustrative embodiment;

FIG. 3 is a flowchart of a process for tracking daily activities utilizing one or more sensor tags in accordance with an illustrative embodiment;

FIG. 4 is a flowchart of a process for activating a sensor tag in accordance with an illustrative embodiment; and

FIG. 5 is a flowchart of a process for communicating data in accordance with an illustrative embodiment;

FIG. 6 is a flowchart of a process for detecting an event in accordance with an illustrative embodiment; and

FIG. 7 depicts a computing system 700 in accordance with an illustrative embodiment.

### SUMMARY

A system, method, and sensor tags for monitoring activities of a user. Sensor tags and a hub in operative communication with the sensor tags are provided. Each of the sensor tags is configured to sense at least one parameter. Each of the sensor tags is associated with one of an item and a user. A first of the sensor tags is positioned on an item no worn by the user. At least one parameter for the sensor tags is communicated to a hub. At least one parameter of the first of the sensor tags is applied to a model using a processor of the hub in order to characterize a user activity. An action is performed based on the user activity characterized using the model. The action includes at least one of storing the user activity on a memory of the hub, communication the user activity from the hub to a remote device, and communicating an alert.

Another embodiment provides a system for determining daily activities of a user. The system includes sensor tags for performing measurements of at least one parameter configured to detect a user, user movements, user biometrics, and environmental conditions. A hub in communication with the sensors tags receives the sensor measurement from the sensor tags and communicates alerts associated with the sensor measurements.

Another embodiment provides a sensor tag. The sensor tag includes a battery powering the sensor tag. The sensor tag includes a logic engine in communication with the battery. The sensor tag includes one or more transceivers in communication with the logic engine for communicating with at least a hub. The sensor tag includes a number of sensors in communication with the logic engine performing sensor measurements. The logic engine determines user activities utilizing the sensor measurements from the sensors and communicates the user activities to a hub associated with the sensor tag utilizing the one or more transceivers.

A system, method, and sensor tags for monitoring activities of a user. Sensor measurements are performed utilizing a sensor array of sensor tags. User activities are detected utilizing the sensor array of the sensor tags. The user activities are communicated to a hub associated with the sensor array of sensor tags.

### DETAILED DESCRIPTION OF THE DISCLOSURE

The illustrative embodiments provide a system, method, sensor, sensor network, and devices for tracking daily activities of a user. The user may represent a patient, resident, employee, or individual. The sensors may represent sensor tags. The sensors may be utilized on fixed objects or structures, objects or structures with limited mobility (e.g., refrigerator, washer, door, handles, etc.), users/pets, mobility devices, and other components or persons. The sensors may be utilized on users, furniture, structures, appliances, or other portions of a home, business, hospitals, facility, or location. For example, common locations for the sensors may include attached or integrated with a belt, watch/wrist band, clothing, wearables, wall, refrigerator, toilet, bed, door, window, mobility device (e.g., wheelchair, walker/rollator, crutches, cane, etc.), pet, or other living or inanimate objects.

The sensors may be configured to take any number of measurements regarding users as well as the applicable environment. For example, the sensors may include accelerometers, gyroscopes, time-of-flight sensors, ambient light sensors, infrared, optical, temperature, barometer, and other applicable sensors. The sensors may be particularly configured to detect motions (or non-motion), activities, falls, or other actions of the user or the lack thereof. As a result, the sensors may track the daily activities of the user.

The sensors include a small profile and shape including a polymer, plastic, metal, rubber, or other housing, support structure, or frame. In one embodiment, the sensors may be disposable after a time period (e.g., six months, one year, 1.5 years). The sensors may also be recharged. For example, the sensors may include a rechargeable battery with a port, contacts, or inductive charging system. The sensors may be recharged when positioned or may be moved or partially moved (e.g., a mounting system may remain while the battery is recharged) to a charging system. The sensors may also include a small solar cell, fuel cell, piezo electric generator, or other power or charging system.

The sensors may be activated or otherwise configured utilizing an electronic device, such as a smart phone, smart wearable, or so forth. For example, a sensor may be paired with a smart phone that utilizes a mobile application to activate the sensor, associate the sensor with one or more users, and provide applicable information as needed. The sensors may communicate with each other as part of a sensor network for a location (e.g., home, residence, facility, etc.). For example, the sensors may form a traditional, relay, or mesh network. The sensors may communicate utilizing one or more transceivers, transmitters, receivers, hybrids, combinations, or so forth that utilize networks or signals, such as Bluetooth low energy, Wi-Fi, ultra-wide band (UWB), Thread, Zigbee, or other communications standards, protocols, or signals. For example, any number of Internet of things networks or communications may be utilized.

The sensors may automatically self-configure themselves based on the positioned location. The self-configuration may be performed utilizing logic including pre-defined algorithms, processes, programs, scripts, data sets, sets of instructions, fixed or programmable logic or so forth. For example, the sensors may utilize first logic for fixed objects in response to determining the sensors do not move. The sensor may utilize additional logic for objects with limited pre-defined motions, such as doors, recliners, toilet handles, windows, appliances or so forth. The sensors may utilize other additional logic for movable devices, such as wheelchairs, walkers/rollators, canes, or so forth. The sensors may utilize other additional logic for users or pets in an applicable environment. In one embodiment, the user may specify a location, device, structure, fixture, object, or user associated with the sensor when activating the sensor, during repurposing or movement of the sensor, or at any other time.

The sensors may detect the daily motions and activity of the user(s) to ensure the well-being and safety of the user. The sensor(s) may also detect a fall utilizing the accelerometers, gyroscopes, lack of motion, location, speed of the user (before and after an event) and utilizing the overall sensors.

The sensors may utilize machine learning, deep learning, artificial intelligence, and historical data and information to better detect user activities over time. The different types of automated learning, such as deep learning may be supervised, semi-supervised, or automatic (unsupervised). Any number of deep neural networks, deep believe networks, recurrent neural networks, and convolutional neural networks may be accessed to perform the processes herein described. For example, the sensors may be adapted to learn the user's activities, actions, and habits over time. The user's activities may even be noted by the user himself/herself (e.g., leave to workout 7:00 a.m. through the garage, breakfast from the refrigerator at 9:00 a.m., shower at 9:30 a.m., nap in the recliner/bed at 11:00 a.m., etc.) for detecting the daily activities and motions of the user.

The sensors may communicate applicable information through one or more hubs in the applicable environment. A dedicated smart hub may receive information from the sensors directly or indirectly (e.g., mesh communications). The user's smart phone, watch, tablet, or other device may also act as the hub for receiving, processing, and/or communicating the information and data from the sensors. Individual or master sensors with or without enhanced memory, transceivers, or capacity may also act as a hub. The sensors may also relay information between themselves to a personal computer, wireless device, network node, or other device that acts as a hub or relays information to a hub (e.g., network connected cloud hub).

The size, shape, and footprint of the sensors may vary based on need, included components, or other technical requirements. For example, the sensors may be square, rectangular, circular, oblong, or may utilize any number of shapes and colors. The sensors may be made to look like decorations or ornaments so that the sensors do not stick out. The sensors may also be integrated into other devices or components, such as doorknobs, outlet/switch covers, molding, hooks, or so forth. The sensors may be attached, fixed, or integrated with any number of living or inanimate objects utilizing adhesives, buttons, hooks/fasteners, magnets, clips, or so forth.

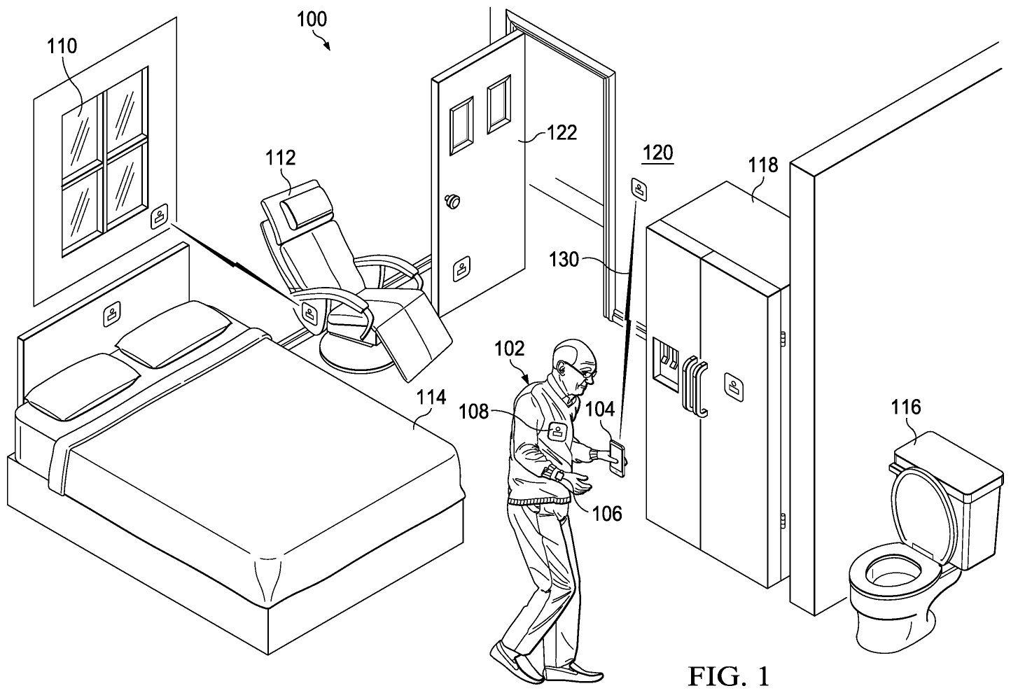

FIG. 1 is a pictorial representation of a sensor environment 100 in accordance with an illustrative embodiment. The sensor environment 100 may represent any number of environments in which a user 102 may live, recreate, reside, work, study, receive treatment, be monitored, or otherwise visit. In one embodiment, the sensor environment 100 may include a user 102 utilizing a wireless device 104. The sensor environment 100 may include any number of sensors 108. The sensors 108 may act independently or as a network. The sensor environment 100 may include objects 109 including a window 110, a chair 112, a bed 114, a toilet 116, a refrigerator 118, a wall 120, and a door 122.

The sensors 108 may track the user 102 throughout the sensor environment 100. In one embodiment, the sensor environment 100 may represent the home of the user 102. For example, the user 102 may be of advanced years, sick, disabled, recovering from an illness/surgery, have disabilities, or otherwise require monitoring. For example, the sensor environment 100 may be equipped, built, or retrofitted with the sensors 108 to ensure the user's safety. The sensors 108 may communicate with each other or with the wireless device 104 or a wearable device 106 utilizing a wireless signal 130.

The sensors 108 may be attached to one or more items, fixtures, or components directly or indirectly. For example, a magnetic connector, housing, or framework may be separately attached to the specified location with the sensors 108 enabled to be removable attached, loaded, or integrated as needed. The sensors 108 may be moved between distinct locations as required for the needs of the user.

The user 102 may be wearing sensors 110. The sensors 110 may be worn on the body or clothing of the user 102. The sensors 110 may also be implanted or otherwise integrated with the body of the user 102. The sensors 110 may represent active or passive devices. For example, the sensors 110 may represent an active device, such as a smart watch/band with at least a battery, transceiver(s), biometric sensors, and logic that both sent and receives wireless signals including applicable information or data. In another example, the sensors 110 may represent a passive device, such as a radio frequency identification (RFID) tag that is activated in response to a wireless signal from the sensor 106 to communicate information or data.

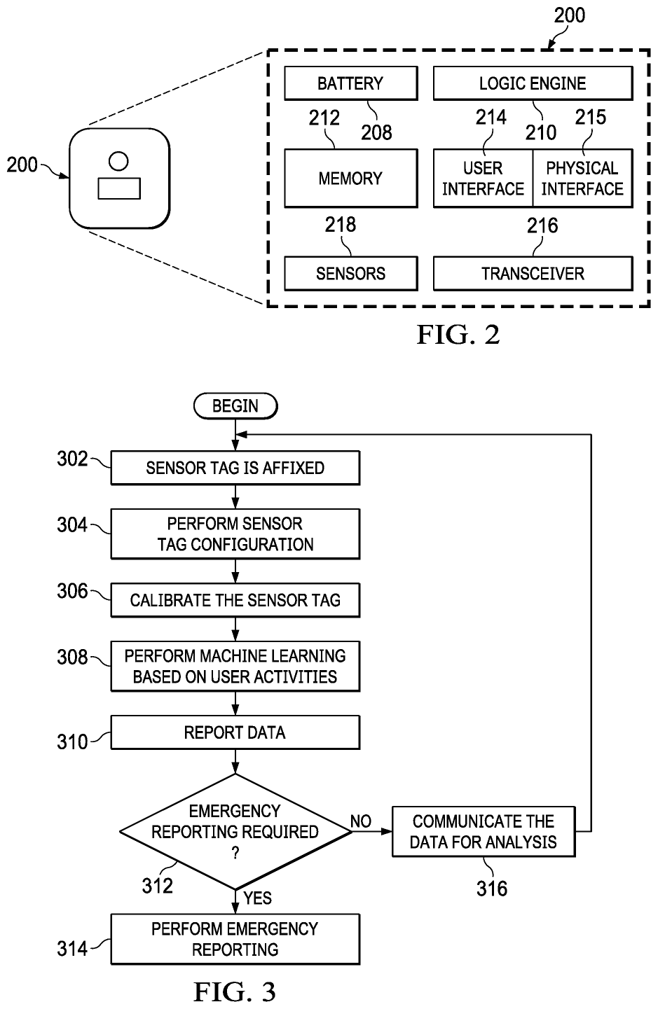

FIG. 2 is a block diagram of a sensor tag 200 utilized as part of a sensor system in accordance with an illustrative embodiment. In one embodiment, the sensor tag 200 may be representative of the sensors 110 of FIG. 1. The sensor tag 200 may represent a standalone sensor that may be connected to users/pets, clothing, wearable devices, walls, furniture, structures, or other living or inanimate components.

The sensor tag 200 may be wirelessly linked to any number of sensors, systems, or devices/wireless devices (not shown), such as the wireless device 104 of FIG. 1. The wireless link may represent location-based, temporary, periodic, or permanent connections, links, signals, exchanges, or communications. For example, wireless devices may represent wearable devices, communications devices, computers, entertainment devices, or so forth. Sensor measurements, user input, selections, and commands may be received from either the sensor tag 200 or the wireless device for processing and implementation.

In some embodiments, the wireless device or other wearable devices may also act as a logging tool for sensor data or measurements made by the sensor tag 200. For example, the wireless device may receive, and share usage data captured by the sensor tag 200 in real-time including user location, position, time standing/laying/sitting, activity performed, environmental data, and so forth. As a result, the wireless device may be utilized to store, display, and synchronize sensor data received or measured by the sensor tag 200.

In one example, the wireless device may store external command profiles for implementing various sensor tag 200 changes or adjustments, reporting or alert processes, or other processes, steps, applications, functions, or so forth. For example, the wireless device may display user pulse rate, temperature, proximity, location, blood oxygenation, distance, calories burned, and so forth as measured by the sensor tag 200 or other sensors (e.g., wearable devices, etc.). The wireless device may also store environmental measurements, spatial information, sound information, and other data regarding known or typical conditions (e.g., temperature, sounds sources, noise levels, environmental conditions, etc.) for specific locations that may be utilized to perform measurements or implementation of the user preferences, settings, and/or parameters. This same information and data may be stored in a memory 212 temporarily or permanently for utilization, logging, historical information, or any number of other uses or purposes. The sensor tag 200 tag may also store all of this information for immediate or subsequent access (e.g., archived information).

In one embodiment, the sensor tag 200 may include a battery 208, a logic engine 210, a memory 212, a user interface 214, a physical interface 215, a transceiver 216, and sensors 218. The sensor tag 200 and the wireless device may have any number of electrical configurations, shapes, and colors and may include various circuitry, connections, and other components utilized to perform the illustrative embodiments.

As previously noted, the sensor tag 200 may be integrated with, attach to, or dock with any number of objects whether living, fixed, or movable. The sensor tag 200 may also be configured to dock with a mount, such as a pocket in smart clothing, a wearable device, furniture, or other preconfigured objects. In one embodiment, the sensor tag 200 may magnetically attach to different components. Adhesives, snap and hook, buttons, magnets, mounts, cradles, ties, or other attachment mechanisms may also be utilized.

The battery 208 is a power storage device configured to power the sensor tag 200. The battery 208 may also represent the power system of the sensor tag 200 that may include plugs, interfaces, transformers, amplifiers, converters, or so forth. In other embodiments, the battery 208 may represent a fuel cell, thermal electric generator, inductive power system, solar cell, ultra-capacitor, or other existing or developing power storage technologies. As a result, the use of the sensor tag 200 may be prolonged. The sensor tag 200 may also be configured to tie into existing power systems utilizing ports, transformers, adapters, interfaces, pins, contacts, inductive interfaces, converters, or so forth.

The logic engine 210 is the logic that controls the operation and functionality of the sensor tag 200. The logic engine 210 may include circuitry, chips, and other digital logic. The logic engine 210 may also include programs, scripts, and instructions that may be implemented or executed to operate the logic engine 210. The logic engine 210 may represent hardware, software, firmware, or any combination thereof.

In one embodiment, the logic engine 210 may include one or more processors. The logic engine 210 may also represent an application specific integrated circuit (ASIC) or field programmable gate array (FPGA). The logic engine 210 may utilize sensor measurements, user input, user preferences and settings, conditions, factors, and environmental conditions to determine the identity of the user, location, position, orientation, movements, and status information (e.g., coming and going information, position in the associated room, etc.), at least in part, from measurements performed by the sensor tag 200.

The sensor tag 200 may identify the user utilizing pre-specified devices, images, biometrics, body composition, or so forth. The identity of the user may be utilized by the logic engine 210 to manage specific configuration of the sensor 200 or associated system. For example, the logic engine 210 may detect information regarding multiple users including an at-risk adult living in a location. The at-risk adult may be monitored more closely or with more sensitive thresholds or criteria in response to determine there are no other users at the location.

In one embodiment, the logic engine may determine spatial information as well as self-configurations and other actions for the sensor tag 200 based on measurements and data from the sensors 218 as well as other connected devices. In one embodiment, the logic engine 210 may manage the calibration and self-configuration based on measurements and data from the sensors 218 as well as other connected devices. The logic engine 210 may also perform any number of mathematical functions (e.g. determining body orientation, average time per location, average rate of movement per minute, hour, day, or year, etc.) to determine or infer the sensor tag 200 configuration, sensor sensitivity, biasing, or adjustments that may be required. The logic engine 210 may utilize wireless signals from other sensors/devices, historical measurements, trends, component degradation or failures, time, and other sensor measurements as causal forces to enhance a mathematical function utilized to perform the determinations, presentation, processing, calculations, and extrapolations performed by the logic engine 210. As previously noted, the sensor tag 200 may detect active or passive signals to detect the presence, position, location, and other applicable information relevant to the user and the user's location, orientation, and movement. For example, the transceiver 216 may communicate a powered signal that is reflected back by the user or a sensor or device worn by the user to provide relevant information.

In one embodiment, the logic engine 210 may calibrate the sensitivity of the sensors 218 for the light conditions at the location based on the time of day. Different locations vary significantly in brightness from morning to night based on windows, paint colors, flooring, orientation, and so forth. The logic engine 210 may calibrate the sensors based on these factors to be able to detect motion of the user. The sensors 218 may distinguish between individuals in the location including a patient, visitors, pets, and so forth. Images, wearable devices, known clothing, or other information may be utilized to identify the user. For example, facial or body recognition may be utilized to identify the users.

The logic engine 210 may also process one or more automated instructions, programs, user preferences, and user input to determine how spatial information is communicated/displayed, utilized, and otherwise implemented by the sensor tag 200. Specific configuration commands or activities may be allowed based on sensor measurements, events, environmental conditions, proximity thresholds, locations, and so forth. For example, the logic engine 210 may implement a spatial information macro allowing the user to view the accrual of biometric information relating to activities, action and movement of the user in real-time or per specified time period. The logic engine 210 may utilize other biometrics, such as exercise, blood pressure, temperature, respiration rate, pulse rate, doctor recommendations, and other applicable information to determine or recommend how much the user should be moving, expected biometrics, expected locations, and other applicable information.

In one example, the spatial information is presented audibly for the user to receive information and make selections. For example, in response to the user approaching the sensor tag 200, the sensor tag 200 may play an audible message indicating “Marian is sitting at 9:45 a.m.” The sensor tag 200 may include a speaker for communicating information, data, and alerts. For example, the speaker may play a message from the logic engine 210 indicating “you have been laying down since 2:00 p.m. and now need to wake up so you can sleep tonight”, “it is time to wake up and do your stretches”, “you should walk your dog”, “you should have a snack”, or other applicable messages. The sensor tag 200 may also pose a question to the user, such as “Have you fallen? or “Are you feeling well?” The information provided by the user may be utilized to send any number of alerts or messages regarding the status of the user.

In one embodiment, a processor included in the logic engine 210 is circuitry or logic enabled to control execution of a set of instructions. The processor may be one or more microprocessors, digital signal processors, application-specific integrated circuits (ASIC), central processing units, or other devices suitable for controlling an electronic device including one or more hardware and software elements, executing software, instructions, programs, and applications, converting and processing signals and information, performing mathematical calculations, and performing other related tasks.

The memory 212 is a hardware element, device, or recording media configured to store data or instructions for subsequent retrieval or access at a later time. The memory 212 may represent static or dynamic memory. The memory 212 may include a hard disk, random access memory, cache, removable media drive, mass storage, or configuration suitable as storage for data, instructions, and information. In one embodiment, the memory 212 and the logic engine 210 may be integrated. The memory 212 may use any type of volatile or non-volatile storage techniques and mediums. The memory 212 may store information related to the user, sensor tag 200, wireless devices, and other peripherals, such as a wireless devices, RFID tags, smart glasses, smart watch, smart clothing, and so forth. In one embodiment, the memory 212 may display or communicate instructions, programs, drivers, or an operating system for controlling the user interface 214 including one or more displays, speakers, tactile generators (e.g., vibrator), and so forth.

The memory 212 may also store user profiles, biometric readings, applications, historical location, position, orientation, and sound information, user input required for self-configuration processes, configuration data (e.g., default, standard, baseline, factory programmed, etc.), user settings and preferences, thresholds, conditions, parameters, signal or processing activity, proximity data, and so forth. The memory 212 may also store pin numbers, passwords, keys, encryption information, network access information, and other information for securely communicating with other sensor tags, networks, wireless devices, and so forth.

The transceiver 216 is a component comprising both a transmitter and receiver which may be combined and share common circuitry on a single housing. The transceiver 216 may communicate utilizing low frequency (LF), high frequency (HF), or ultra-high frequency (UHF), radio frequency identification (RFID), near field communications (NFC), near-field magnetic induction (NFMI) communication, Bluetooth, Wi-Fi, ultra-wide band, ZigBee, Ant+, near field communications, wireless USB, infrared, mobile body area networks, ultra-wideband communications, cellular (e.g., 3G, 4G, 5G, PCS, GSM, etc.), infrared, or other suitable radio frequency standards, networks, protocols, or communications. For example, the transceiver 216 may coordinate communications and actions between the sensor tag 200 and one or more devices, tags, or sensors utilizing radio frequency communications. The transceiver 216 may also be a hybrid transceiver that supports a number of different communications. The transceiver 216 may also detect time receipt differentials, amplitudes, and other information to calculate/infer distance between the sensor tag 200 and external devices, such as tags, smart watches, wireless device or other sensors. The transceiver 216 may also represent one or more separate receivers and/or transmitters.

The components of the sensor tag 200 may be electrically connected utilizing any number of wires, contact points, leads, busses, chips, wireless interfaces, or so forth. In addition, the sensor tag 200 may include any number of computing and communications components, devices or elements which may include busses, motherboards, circuits, chips, sensors, ports, interfaces, cards, converters, adapters, connections, transceivers, displays, antennas, and other similar components.

The physical interface 215 is hardware interface of the sensor tag 200 for connecting and communicating with computing devices (e.g., desktops, laptops, tablets, gaming devices, etc.), wireless devices or other electrical components, devices, or systems. In one embodiment, the physical interface 215 may include power, communications, wireless, and other ports and interfaces. For example, synching and charging may be performed by an external device through the physical interface 215.

The physical interface 215 may include any number of pins, arms, or connectors for electrically interfacing with the contacts or other interface components of external devices or other charging or synchronization devices. For example, the physical interface 215 may include USB, HDMI, Firewire, micro USB, and AC/DC ports and interfaces. In one embodiment, the physical interface 215 is a magnetic interface that automatically couples to contacts or an interface of the sensor tag 200 for powering the components of the sensor tag 200 or recharging the battery 208. A sealed interface may be utilized to ensure that the sensor tag 200. In another embodiment, the physical interface 215 may include a wireless induction device for recharging or

The user interface 214 is a hardware and software interface for receiving commands, instructions, or input through touch (haptics) of the user, voice commands, or predefined motions. For example, the user interface 214 may include a button for activating the sensor tag 200 (e.g., turning the sensor tag 200 on and off). One or more buttons may also be utilized to activate different modes, sensor configurations (e.g., bed, window, door, walk, user, etc.), or provide other applicable information. The user interface 214 may also include a touch screen (including a fingerprint scanner), one or more cameras or image sensors, microphones, speakers, and so forth. Although not shown, the sensor tags may also include one or more speakers and speaker components (e.g., signal generators, amplifiers, drivers, and other circuitry) configured to generate sounds waves at distinct frequency ranges (e.g., bass, woofer, tweeter, midrange, etc.) or to vibrate at specified frequencies to be perceived by the user as sound waves.

The user interface 214 may be utilized to control the other functions of the sensor tag 200. The user interface 214 may include the hardware buttons, one or more touch sensitive buttons or portions, a miniature screen or display, or other input/output components. The user interface 214 may be controlled by the user or based on commands received from an associated wireless device, or other authorized devices. The user may also establish or cancel self-configuration of the sensor tag 200 utilizing the user interface 214.

The user interface 214 may also include one or more microphones. The microphone(s) may represent any number microphone types utilized to sense the user's voice, external noise, and so forth. The microphones may be utilized to receive user input as well as detect the presence of the users (the microphones may also be part of the sensors 218). For example, the speaker and microphones may be utilized to ask the user if she is okay after an event is detected (e.g., impact, no movement within a threshold based on the time of day, remaining in an unexpected location, etc.)

The user interface 214 may include any number and type of devices for receiving user input and providing information to the user. In one example, the device includes a tactile interface, an audio interface, and a visual interface. The tactile interface includes features that receive and transmit via touch. For example, as noted above, the sensor may include one or more buttons to receive user input. In one example, a single button of the sensor tag 200 may identify the user utilizing a fingerprint scan as well as recording a time that the user is at an associated location. Another selection of the button may indicate that the user is leaving the associated location. Buttons, switches, or other components on the sensor may also control emergency messages that may be sent based on being pressed or activated.

The audio interface portion of the user interface 214 may include any suitable speaker and/or microphone, as known in the art. For example, a speaker may be as part of the housing of the sensor tag 200 and programmed to emit information in the form of audio output. The microphone may also receive input from the user if the user has fallen.

In one embodiment, the biometric, private, and other secured data of the user may be encrypted and stored within a secure portion of the memory 212 to prevent unwanted access or hacking. The sensor tag 200 may also store important user profile and biometric data, such as medical information (e.g., medical conditions, logged biometrics, contacts, etc.) and identifying biometric information, for sharing in response to an emergency or authenticated request. The sensor tag 200 may be utilized with other sensor tags in an array (one or more) to form a system. In one embodiment, the sensor tag 200 may be a master sensor tag that the other sensor tags communicate with to report data and information. For example, the master sensor tag may include a transceiver 216 configured for making cellular calls, communicating through Wi-Fi, or so forth. For example, the sensor tags in a location may communicate with each other utilizing Bluetooth low energy and the master sensor tag may communicate with a hub, wireless device, or tower utilizing a cellular, Wi-Fi, ultra-wide band, or other longer-range connection. For example, a mesh network may be established between devices in a large home or commercial building where distances are to great for all sensor tags to communicate to a central location.

The sensors 218 may include photodetectors, miniature cameras, microphones, accelerometers, gyroscopes, impact/force detectors, thermometers, inertial sensors, and other similar instruments for detecting the user's status, position, orientation, motion, and environmental conditions. The sensors 218 may also be utilized to determine the biometrics, activities, locations, other users in the environment, animals, devices, and so forth. In one embodiment, the sensors 218 may store data that may be shared with other components (e.g., logic engine 210 implementing a configuration process), users, and devices. For example, the sensors 218 may detect when the user is standing, sitting, laying down, walking, running, and so forth. The sensors 218 may also detect the proximity of the user to the sensor tag 200.

The sensors 218 may also include photodetectors, ultrasonic mapping devices, or radar that scan the body and body parts of the user when positioned for utilization. The mapping may also extend to the environment of the user. The topographical image may also be utilized to perform additional analysis based on the determined position, orientation, and determinations of the sensors 218.

The sensors 218 may pass measurements, readings, and data about the user and environment to the logic engine 210 for performing configuration processes and algorithms. Likewise, the memory 212 may store the location detection, sound processing and configuration programs, algorithms, steps, baseline data, sensor measurement data, and so forth. This data and information may also be communicated to a connected device for storage or analysis. The sensor measurements may be compared against the baseline data to determine variations and how to compensate or adjust the sensor tag 200 based on the sensor 218 measurements. The logic engine 210 may also perform pattern analysis with the sensor measurements to calibrate or tune the sensors 218 based on established patterns, historical data, or information.

Externally connected hubs, tags, chips, sensors, or wireless devices may include components similar in structure and functionality to those shown for the sensor tag 200. For example, a hub or wireless device may include any number of processors, batteries, memories, busses, motherboards, chips, transceivers, peripherals, sensors, displays, cards, ports, adapters, interconnects, sensors, and so forth. In one embodiment, the hub or wireless device may include one or more processors and memories for storing instructions. The instructions may be executed as part of an operating system, application, browser, or so forth to implement the features herein described. For example, the user may set preferences for the sensor tag 200 to communication information, perform processes and analysis, and self-configure based on specified events, locations, activities, or user input. Likewise, the preferences may manage the actions taken by the sensor tag 200 in response to identifying activities of specific users (e.g., low risks users may not be monitored closely).

The sensor tag 200 may also execute an application with settings or conditions for communication, self-configuration, updating, synchronizing, sharing, saving, identifying, calibrating, and utilizing biometric and environmental information as herein described. For example, alerts may be sent to the user to stand up or otherwise move in response to the user sitting for an hour of time (e.g., the user may be prone to cramps, blood clots, or other conditions that require movement). The alert may be communicated through a text message, in-application message communicated through the user's computer, smart phone, smart watch, smart hub, or other device, audio alert from the user interface 214, vibration, flashing lights, display, or other system for the sensor tag 200. In another example, the sensor tag 200 may provide a warning if the user is swaying or may fall. Likewise, alerts may be sent to predetermined parties if the user does fall.

In some embodiments, the sensor tag 200 may be attached to a walker, rollator, furniture, cabinets, or other devices for determining that the user is moving about a home, apartment, care facility, office, or other location. For example, the user may wear an RFID tag that may be detected as the user moves throughout a location as well as any potential fall events. In one embodiment, the sensor tag 200 may detect unexpected separations from an RFID tag to determine a fall event has happened. For example, the detected range between the two devices may suddenly change from two feet to four feet with the RFID tag well below the sensor tag 200 indicating an unwanted event has happened.

As originally packaged, the sensor tag 200 may include peripheral devices such as charging cords, power adapters, inductive charging adapters, solar cells, ambient light chargers, batteries, lanyards, additional light arrays, speakers, smart case covers, transceivers (e.g., Wi-Fi, UWB, cellular, etc.), or so forth.

FIG. 3 is a flowchart of a process for tracking daily activities utilizing one or more sensor tags in accordance with an illustrative embodiment. Although described for a single sensor tag, the process of FIGS. 3-6 may be performed by one or more sensors in a sensor network or system (referred to for simplicity as a “system”). The sensors may also communicate with one or more hub devices (e.g., smart phone, master sensor, smart wearable, tablet, computer, etc.).

The process may begin by determining a sensor tag is affixed (step 302). The sensor tag may be connected utilizing integrated or separately attached adhesives, clips, buttons, mounts, ports, clamps, docking modules, pockets, hook and loop (i.e., Velcro), or other connections mechanisms or means. The sensor tag may also be built-in or integrated with an associated item, such as clothing, straps, wearables, furniture, appliances, structures, or so forth. The sensor tag may be internally or externally powered. In one embodiment, the sensor tag may be inductively powered or powered through a port or connector.

Next, the system performs sensor tag configuration (step 304). The configuration process of step 304 may be an automated, semi-automatic, or manual process. During step 304, the user or the sensor tag itself may select an appropriate program, process, or algorithm associated with the location, item, or user to which the sensor tag is affixed. For example, different sensory processes may be implemented for a user wearing the sensor tag as compared to sensor tags that are attached to a door, bed, or toilet handle. For example, accelerometer readings may not be performed or prioritized for a device attached to a bed or wall. The sensor tag may expect different types of sensor measurements to be more important based on the location and utilization of the sensor tag. The sensor tags may be specially designed to track the daily activities of the user but may also provide an early warning system for emergencies, such as fires, flooding, disturbances, or so forth.

In one embodiment, during step 304 the sensor tag may be activated by pairing or communication with a wireless device, smart wearable device, or other computing or communications device. For example, a mobile application executed by a smart phone may allow the user to pair the smart phone with the sensor tag. A user interface may then allow the user to specify the location, utilization, and/or variables of the sensor tag from a drop-down menu for all known uses of the sensor tags (e.g., structures, doors, windows, walkers, furniture, users, pets, etc.). In one embodiment, the sensor tag may automatically configure itself immediately or over-time. For example, the sensor tag may determine whether it moves or is fixed. The sensor tag may also determine whether motions are defined or random. For example, doors, refrigerators, windows, or other devices may have define or fixed motions that are known and recorded. Other devices, such as walkers/rollators, canes, wheelchairs, may have random motions. Similarly, individual users or pets may move randomly even though some patterns and behaviors may be recorded.

Next, the system calibrates the sensor tag (step 306). The sensor tag may be calibrated based on the configuration information and data determined during step 304. For example, sensor tags may determine which sensors within the senor tag will be most useful and beneficial based on the location. For example, for a hallway, proximity sensors may be the most important. The sensor tags may also determine default information, such as available view/sensor range, temperature, proximity to other items and devices, barometric readings, ambient light, impacts/speeds, and other applicable information. The calibration information and data may be utilized to best measure variations from the standard/default measurements to make more accurate determinations regarding the status of the user and/or environment. The calibration process of step 306 may be performed in conjunction with measurements from other sensor tags that may be part of the system/network. For example, relevant information and data applicable to the user and/or environment may be shared between sensor tags (e.g., common movements, typical temperature, stature/height/weight/build, skin tone, voice profile, etc.). The sensor tag may perform a calibration process for the various sensors. The calibration may be performed based on historical information, bias levels, and so forth. The calibration process may also include a reboot or reset. Various thresholds may be utilized to perform fall risk prediction and detection. In one embodiment, the sensors may communicate an alert indicating that fall likelihood has surpassed one or more levels, percentages, or so forth. In another embodiment, the system may communicate one or more alerts indicating that a fall or other negative event has happened to one or more specified users, devices, systems, applications, or so forth.

In one embodiment, a profile may be uploaded to the sensor tags based on their specified location (e.g., wall, chair, refrigerator, garage, car, desk, recliner, table, sink, bed, dresser, ceiling, etc.). The profile or model may specify what sensors are used by the sensor tag. For example, based on the location, all or a portion of the sensors may be utilized to preserve the battery life and avoid sending unnecessary data. For example, a hallway may use of the sensors on the sensor tag wherein the garage may only utilize motion detection.

Next, the system performs machine learning based on user activities (step 308). The machine learning, behavior processing, and artificial intelligence may also include documenting the historical sensor measurements and trends measured by the sensor tag. The machine learning process may note sensor measurements that are anticipated or otherwise expected based on trends in recent days, weeks, months, or years. The machine learning may be performed by the system or may be communicated to external systems or devices, such as servers, computing devices, or cloud system/networks. In one embodiment, the sensor tags for a location may communicate with a hub. The hub may communicate the applicable data and information from the sensor tags to a central location to perform analysis, processing, and machine learning as noted in step 308. As previously noted, the hub may represent a master sensor tag, wireless device, computing device, or other applicable device that is integrated with, located within or near, or otherwise communicates with the system. The machine learning may learn activities of the user and associate those activities with locations, body position/orientation, expected time-of-day, associated user activities, and so forth. As a result, the system may be able to determine if the user has been in a recliner or bathtub too long to check the status of the user. Similarly, any sensor measurements indicating that the user is or may be laying on the ground outside of a known stretching area may automatically generate an alert.

Next, the system reports data (step 310). The data may include sensor measurements, data, and information captured by the system as well as any other devices authorized to communicate with the system (e.g., wearables, implantable devices, smart clothing, security systems, etc.). The data may be communicated during step 310 to one or more sensor tags, wireless devices, hubs, computers, or other applicable devices. The data may be communicated utilizing any number of communications signals, standards, or protocols. For example, Bluetooth, Bluetooth Low Energy (BLE), Wi-Fi, ultra-wide band, Zigbee, infrared, Thread, EnOcean, Wi-SUN, or other available or developing wireless technologies.

Next, the system determines whether emergency reporting is required (step 312). The report data may be analyzed utilizing the devices of the system or externally based on available resources. The system may utilize any number of thresholds, criteria, parameters, or other information. For example, different parameters may be utilized for different types of sensor measurements including high/low temperature of the user/environment, impacts of the user/devices, excessive motion or lack of motion (e.g., seizure, stroke, fainting, etc.), position/orientation of the user (e.g., lying is a position where standing only is expected such as in the kitchen), and time periods associated with any of these measurements.

If emergency reporting is required during step 312, the system performs emergency reporting (step 314). The emergency reporting may occur sequentially, concurrently, or simultaneously to any number of parties. For example, in a care facility, nurses may be first notified of a potential emergency followed by a 911 call. In one embodiment, emergencies may be verified or cleared from the system by authorized parties. For example, mobile applications may be utilized to access a profile or location associated with the user to verify or deny an applicable emergency.

If emergency reporting is not required during step 312, the system communicates the data for analysis (step 316). The additional analysis may include machine learning, big data analytics, trend determination, and any number of mathematical and statistical analysis.

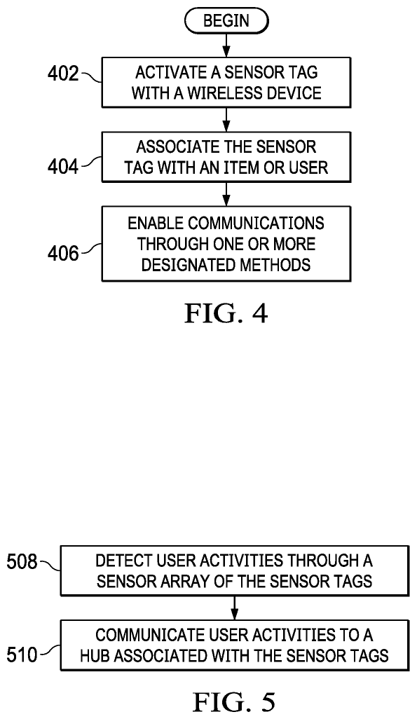

FIG. 4 is a flowchart of a process for activating a sensor tag in accordance with an illustrative embodiment. In one embodiment, the process of FIG. 4 may be added to or performed as part of the process or steps of FIG. 3 or 5, such as step 304 of FIG. 3.

In one embodiment, the process may begin by activating a sensor tag with a wireless device (step 402). The sensor tag may include one or more buttons, switches, touch sensors, dials, or other hardware/firmware/software interfaces for interacting with the user. A battery may also be inserted or activated during the process of step 402. In one embodiment, a user interface available through a website or application may be utilized to both send, receive, and display applicable information regarding the sensor tag(s), system, data, and so forth. For example, the user may view performance, battery status, reported/logged data, alerts, and other applicable information utilizing the system.

Next, the system associates the sensor tag with an item or user (step 404). In one embodiment, the user may select the item or user the sensor tag is attached to or associated with. For example, the sensor tag may be associated with a door, wall, window, bed, bathroom, kitchen island, recliner, stairs, or so forth. The sensor may be paired with one or more user profiles in response to a smart phone executing a mobile application for managing the sensors being positioned within ten feet of the sensor. Each of the sensors may be associated with a location (e.g., home, facility, residence, hospital, nursing home, care center, etc.) or one or more users (e.g., an individual, aging couples, families, etc.). The association of the sensor tag with one or more users is important for accurately processing data, performing reporting, and communicating alerts.

Next, the system enables communications through one or more designated methods (step 406). As previously noted, the designated methods may include communications with a wireless or computing device, communications through a network (e.g., Wi-Fi, ultra-wide band, cellular, mass, etc.), one or more of direct sensor to sensor communications, communications through a hub, powerline communications, or other forms of communications. The different types of communications may be performed automatically or in response to user input through a browser/application executed by an electronic device. For example, the user may be required to provide network names, access keys, or passwords for accessing one or more networks available at the location. The sensor tags may also form a mesh network to communicate with a hub, wireless device, router, or other communications device that may not be accessible to all of the sensor tags. In one embodiment, the system may include a master sensor tag with enhanced memory and logic for storing sensor measurements made throughout the system. The master sensor tag may also perform analysis, machine learning, deep learning, communication management, and alert generation.

During step 406, the user may also specify individuals, groups, parties, or others that receive communications and alerts from the sensor tag and associated system. For example, communications preferences may be utilized to specify how, when, and in what priority communications and alerts are sent by the system or individual sensor tags. The communications preferences may also specify the types of communications that are utilized including in-app messages, texts, emails, automated phone calls, courier messages, or so forth.

In one embodiment, the sensor tags may be configured to plug into wall outlets of the location. The sensor tags may then utilize the electricity available through the wired systems of the location to perform continuous monitoring without concern for depleting batteries or other resources of the sensor tag. The sensor tags may also perform powerline communications with one or more dedicated devices.

FIG. 5 is a flowchart of a process for communicating data in accordance with an illustrative embodiment. The process may begin by detecting user activities through a sensor array of the sensor tags (step 508). The multiple sensor tags may be attached to a user or item (e.g., wall, furniture, floor, appliance, mirror, etc.). The sensor tags may each measure one or more parameters. For example, the sensor tags may sense or capture movement, images (e.g., facial, body recognition, etc.), light changes, vibrations, pressure differentials (e.g., air, water, etc.), sounds, touch, The sensor tag may be configured to utilize one or more of the sensors of the sensor array based on the location and configuration of the applicable sensor tag. For example, sensor tags that are affixed to items that do not move may utilize the integrated accelerometers and very limited fashion (e.g., detecting a user fall against the item, detecting a lack of vibrations indicating that someone has left the chair/bed/bathtub, etc.). The sensor tags may rely much more on infrared/optical sensors and the other suite of sensors included in the sensor array. The user activities may represent any number of actions, processes, steps, routine, movements, or other activities of the user. As previously noted, the user may sometimes represent a patient, resident, or individual requiring special observation, monitoring, and tracking.

Next, the system communicates user activities to a hub associated with the sensor tags (step 510). The sensor tags may send the sensor measurements or parameters as detected in raw format or may send them as user activities (e.g., processed information). In one embodiment, the sensor tags may include a memory or cache for temporarily storing captured information and data. For example, the sensor tags may include sufficient memory to capture sensor readings for hours, days, or weeks before the oldest data is automatically deleted or replaced with newly captured sensor information and data. In one embodiment, the hub is a dedicated device configured to receive information from the sensor tags, wearable devices (e.g., smart bands, smart watches, headbands, smart clothing, etc.), implants, external/third-party sensors, and other devices that capture information about the user, environment of the user, and/or location.

The hub may apply machine learning, artificial intelligence or deep learning to the information and data received from one or more sensor tags to determine the user activities. The hub may utilize the location, position/orientation, vibrations, light, images, motion, historical readings, expected activities, and other information for the sensor tag/user. For example, the sensor measurements may be characterized by the hub as sleeping, walking, cooking, using the restroom, bathing, reading, fallen, in distress, confused/lost, or so forth. The hub may include settings, parameters, or configuration by an administrator, caregiver, parent/guardian, medical professional, or other to define what parameters and activities are considered acceptable and normal and what activities may trigger one or more alerts.

As noted, the sensor tags may communicate sensor measurements (raw or processed) or the user activity. The sensor tags or the hub may determine the user activity based on the sensor measurements. The hub may process the information and data from the sensor tags to perform any number of actions. In one embodiment, the hub may utilize a model developed based on default settings, machine learning, and approved conditions, parameters, and activities to storing the user activity on a memory. For example, the user activity may be saved for subsequent reference to determine when there may be an actual emergency. The hub may also communicate the user activity to a remote device or user. The user activity may be sent as a status check, to document status, for insurance purpose, for peace of mind, or to otherwise monitor the well-being, safety, condition, and status of the user. The hub may also communicate an alert. The alert may be sent in response to determining the status, condition, position, location, orientation, behavior, sounds, biometrics, or other data and information associated with the user does not fit within a standard or acceptable pattern. The alert may be sent to one or more authorized users, such as family, friends, caregivers, medical professionals, professional monitoring services, emergency services, or so forth.

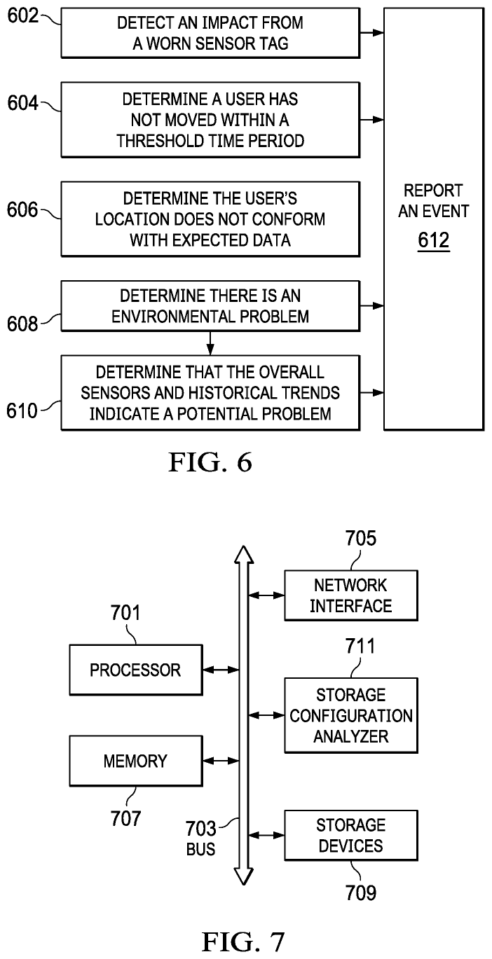

FIG. 6 is a flowchart of a process for detecting an event in accordance with an illustrative embodiment. The process of FIG. 6 may be performed by the system or one or more of the sensor tags acting independently. In one embodiment, steps 602-610 may be performed simultaneously, concurrently, or in any order to detect an applicable event. The determinations, processes, or analysis of steps 602-610 may be performed at any time. The event may be a falling event, medical event (e.g., heart attack, stroke, seizure, etc.), or other negative event that may require the user to receive a visit, status verification, help, assistance, or medical treatment. One or more of steps 602-610 may be utilized individually or in combination to determine an event is occurring.

The system may detect an impact from a worn sensor tag (step 602). The sensor tag may be affixed to the clothing, body, accessories, or other items worn, on, or proximate the body of the user. The impact may be detected utilizing one or more accelerometers. During step 602, the system may also determine the orientation of the user's body utilizing the accelerometer or one or more gyroscopes, magnetometers, or other applicable sensors.

The system may also determine the user has not moved within a threshold time period (step 604). The determination of step 604 may be based on the location of the user and time of day. For example, thresholds utilized by the system for non-activity may be significantly longer for a bed of the user during normal sleeping hours. The thresholds for the user in the kitchen or bathroom may be substantially less.

The system may also determine that the user's location does not conform with expected data (step 608). The user may have a number of expected locations where significant periods of time are spent, such as their bed, a recliner, a kitchen table, a bathtub/shower, and other locations. The resolution of the sensor tags may vary. In one embodiment, the sensor tags may be able to determine the user's exact location and orientation within a resolution of inches or a foot. The resolution of the sensor tags may also vary greater or less. For example, the sensor tags may detect that the user is lying on the kitchen floor which should report an event (step 612). The sensor tags may determine the user's location does not conform if the location is unusual for the user. The sensor tags may also use factors, such as the time in the location, the position/orientation of the user, and other discernable information. The sensor tags may act as beacons for detecting the presence of the user.

The system may also determine there is an environmental problem (step 608). The environmental problem may indicate conditions are unacceptable for the well-being of the user. For example, the sensor tags may detect conditions, such as fires, noxious gasses (i.e., carbon monoxide, natural gas, etc.), flooding, unexpected wind, temperatures past a threshold (e.g., hot or cold), or so forth. The environmental problem may indicate that the user is in danger or in an undesirable situation.

The system may also determine that the overall sensors and historical trends indicate a potential problem (step 610). Sensor measurements regarding the user biometrics, location/orientation, movements, lack of movement, and other relevant information may be utilized together. The information and data from steps 602-608 may be utilized alone or in combination to both detect and report an event (step 612).

During step 612, any number or type of messages may be communicated. The system may also determine whether emergency report is required. If needed, emergency reporting may be communicated to 911 or other emergency services. During step 612, the event may also be reported utilizing a designated communications plan. The communications plan may specify the order in which communications are sent to systems, devices, users (i.e., family, friend, assigned medical professionals, caregivers, etc.), entities, organizations, or others.

In one embodiment, the sensor tags may include microphones and speakers for audibly verifying the status of the user. For example, the sensor tag most closely positioned to the user may audibly ask “is everything okay.” The microphone of the sensor tag may also listen for a response indicating whether the user is fine or whether an alert or other communication need to be sent.

The information and alerts may be communicated to the user and/or any number of specified devices/users. The usage information may be communicated based on any number of thresholds. The alerts may indicate if the user has fallen, not moved within a specified time period, or based on other biometrics measured by the sensor or other associated sensors (e.g., the wireless device).

The sensor tags may communicate with wireless devices including one or more biosensing wearable devices (e.g., helmets, hearing aids, stickers, bands, sensor packages, hearables, etc.), smart phones, web interfaces, or so forth. For example, the microphones, speakers, accelerometers, timers, gyroscopes, magnetometers, thermometers, and other components of the biosensing wearable may be utilized to determine biometric information. The processes of FIGS. 3-6 may be performed automatically or utilizing user input, interactions, or feedback. The system may utilize physiological information regarding the user to set thresholds, monitor the user, analyze information and data, and generate alerts. The user physiology may include height, weight, activity, body dimensions, symmetry, and size, dominant hand, status, diseases, medical conditions, and age of a user/patient. The physiological parameters may be determined from user input, application utilization, measurements, data from the wireless device, a user profile, medical information, or so forth.

The system and sensor tags of the illustrative embodiments are configured to detect, measure, analyze, and alert the user and others regarding potential falling events or other issues. The senor tags may be integrated with or communicated with standard wearable and wireless devices (e.g., smart watches, bands, smart clothing, jewelry, smart phones, etc.). The captured information and data may be analyzed, processed, displayed, and communicated to the user and other applicable parties. The status and reporting information of the system may be reported through in-app, browser, emails, text messages, or other applicable messages.

The illustrative embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, embodiments of the inventive subject matter may take the form of a computer program product embodied in any tangible medium of expression having computer usable program code embodied in the medium. The described embodiments may be provided as a computer program product, or software, that may include a machine-readable medium having stored thereon instructions, which may be used to program a computing system (or other electronic device(s)) to perform a process according to embodiments, whether presently described or not, since every conceivable variation is not enumerated herein. A machine-readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions. In addition, embodiments may be embodied in an electrical, optical, acoustical or other form of propagated signal (e.g., carrier waves, infrared signals, digital signals, etc.), or wireline, wireless, or another communications medium.

Computer program code for carrying out operations of the embodiments may be written in any combination of one or more programming languages, including an object-oriented programming language such as Java, Smalltalk, C++ or the like and conventional procedural programming languages, such as the “C” programming language or similar programming languages. The program code may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN), a personal area network (PAN), or a wide area network (WAN), or the connection may be made to an external computer (e.g., through the Internet using an Internet Service Provider).

The illustrative embodiments may be implemented by an independent cloud system or network or any number of smart devices (e.g., centralized computers, servers, application networks, etc.). Historical information across patients, facilities, and organizations may be processed without identifying information to better perform analysis, predictions, assign values, calculate scores, establish thresholds for detecting falls and other unwanted patient events, and share data. FIG. 7 depicts a computing system 700 in accordance with an illustrative embodiment. The computing system 700 may be representative of all or portions of the sensor tag or hub of FIGS. 1 and 2. The computing system 700 includes a processor unit 701 (possibly including multiple processors, multiple cores, multiple nodes, and/or implementing multi-threading, etc.). The computing system includes memory 707. The memory 707 may be system memory (e.g., one or more of cache, SRAM, DRAM, zero capacitor RAM, Twin Transistor RAM, eDRAM, EDO RAM, DDR RAM, EEPROM, NRAM, RRAM, SONOS, PRAM, etc.) or any one or more of the above already described possible realizations of machine-readable media. The computing system also includes a bus 703 (e.g., PCI, ISA, PCI-Express, HyperTransport®, InfiniBand®, NuBus, etc.), a network interface 705 (e.g., an ATM interface, an Ethernet interface, a Frame Relay interface, SONET interface, wireless interface, etc.), and a storage device(s) 709 (e.g., optical storage, magnetic storage, etc.). The system memory 707 embodies functionality to implement embodiments described above. The system memory 707 may include one or more functionalities that store personal data, parameters, application, user profiles, and so forth. Code may be implemented in any of the other devices of the computing system 700. Any one of these functionalities may be partially (or entirely) implemented in hardware and/or on the processing unit 701. For example, the functionality may be implemented with an application specific integrated circuit, in logic implemented in the processing unit 701, in a co-processor on a peripheral device or card, etc. Further, realizations may include fewer or additional components not illustrated in FIG. 7 (e.g., video cards, audio cards, additional network interfaces, peripheral devices, etc.). The processor unit 701, the storage device(s) 709, and the network interface 705 are coupled to the bus 703. Although illustrated as being coupled to the bus 703, the memory 707 may be coupled to the processor unit 701.

The features, steps, and components of the illustrative embodiments may be combined in any number of ways and are not limited specifically to those described. In particular, the illustrative embodiments contemplate numerous variations in the smart devices and communications described. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of embodiments, processes or methods of the invention. It is understood that any other modifications, substitutions, and/or additions may be made, which are within the intended spirit and scope of the disclosure. For the foregoing, it can be seen that the disclosure accomplishes at least all of the intended objectives.

The previous detailed description is of a small number of embodiments for implementing the invention and is not intended to be limiting in scope. The following claims set forth a number of the embodiments of the invention disclosed with greater particularity.

What is claimed is:

1. A method for monitoring activities of a user, the method comprising:
providing a plurality of sensor tags and a hub in operative communication with the plurality of sensor tags, each of the plurality of sensor tags is configured to sense at least one parameter;
associating each of the plurality of sensor tags with one of an item and a user, wherein a first of the plurality of sensor tags is positioned on an item not worn by the user;
sensing the at least one parameter for the plurality of sensor tags;
communicating the at least one parameter for the plurality of sensor tags to a hub;
applying at least the at least one parameter of the first of the plurality of sensor tags to a model using a processor of the hub in order to characterize a user activity; and
performing an action based on the user activity characterized using the model, wherein the action comprises at least one of storing the user activity on a memory of the hub, communicating the user activity from the hub to a remote device, and communicating an alert.

2. The method of claim 1, wherein a second of the plurality of sensor tags is worn by the user and senses at least a second parameter that is included with the at least one parameter.

3. The method of claim 1, further comprising:
calibrating each of the plurality of sensor tags for the item and the user.

4. The method of claim 1, further comprising:
performing machine learning based on the user activities.

5. The method of claim 1, wherein the communicating further comprises:
determining whether there is an emergency in response to 1) an impact of the user, 2) user movement exceeding a threshold, 3) a location of the user not conforming with expected data, 4) determining there is an environmental problem in the location of the user, and 5) determining that overall sensors and historical trends indicate a potential problem.

6. The method of claim 1, further comprising:
determining at least a position and an orientation of the user.

7. The method of claim 1, wherein the alert is one or more emergency alerts send to one or more users and devices that are authorized to receive the alert.

8. The method of claim 5, wherein the environmental problem includes one or more of the heat, cold, fire or smoke, and flooding.

9. The method of claim 1, further comprising:
activating each of the plurality of sensor tags;
associating each of the plurality of sensor tags with an item or user; and
enabling communications of the plurality of sensor tags.

10. The method of claim 9, wherein the activating is performed utilizing a wireless device, and wherein the communications of the sensor tags are performed through a network, utilizing a mesh network of the sensor tags, or communications with the wireless device.

11. A system for determining daily activities of a user, comprising:
a plurality of sensor tags performing sensor measurements configured to detect a user, user movements, user biometrics, and environmental conditions;
a hub in communication with the sensor tags receives the sensor measurements from the plurality of sensor tags and communicating alerts associated with the sensor measurements.

12. The system of claim 11, wherein the plurality of sensor tags are controlled by an electronic device in communication with one or more of the plurality of sensor tags.

13. The system of claim 11, wherein the plurality of sensor tags and the hub communicate utilizing a mesh network.

14. The system of claim 11, wherein one or more of the plurality of sensors include a logic engine in communication with the plurality of sensors that analyze the measurements and communicate status of the user to one or more designated parties.

15. The system of claim 11, determining whether there is an emergency in response to 1) an impact of the user, 2) user movement exceeding a threshold, 3) a location of the user not conforming with expected data, 4) determining there is an environmental problem in the location of the user, and 5) determining that overall sensors and historical trends indicate a potential problem.

16. The system of claim 1, wherein sensor tag is activated utilizing a wireless device executing an application for managing the sensor tag, wherein the sensor tag is associated with an item or user, and wherein communications are enabled through one or more devices.

17. The system of claim 14, wherein the logic engine communicates an alert to the one or more designated parties, wherein the plurality of sensors are attached to one or more pieces of furniture, mobility devices, and a user.

18. A sensor tag, comprising:
a battery powering the sensor tag;
a logic engine in communication with the battery;
one or more transceivers in communication with the logic engine for communicating with at least a hub;
a plurality of sensors in communication with the logic engine performing sensor measurements, wherein the logic engine determines user activities utilizing the sensor measurements from the plurality of sensors; and communicate the user activities to a hub associated with the sensor tag utilizing the one or more transceivers.

19. The sensor tag of claim 18, wherein set of instructions are further executed to:
activate the sensor tag utilizing a wireless device executing an application for managing the sensor tag;
associate the sensor tag with an item or user; and
enable communications with the sensor tag through one or more devices.

20. The sensor tag of claim 18, wherein the set of instructions determine whether there is an emergency in response to 1) an impact of the user, 2) user movement exceeding a threshold, 3) a location of the user not conforming with expected data, 4) determining there is an environmental problem in the location of the user, and 5) determining that overall sensors and historical trends indicate a potential problem.
